(19) United States
(12) Patent Application Publication
(10) Pub. No.: US 2016/0158424 A1
(43) Pub. Date: Jun. 9, 2016

(54) HIDDEN AND WEARABLE BREAST-MILK PUMPING SYSTEM

(71) Applicant: Metal Industries Research & Development Centre, Kaohsiung (TW)

(72) Inventors: Hui-Ching Chen, Taipei City (TW); Chia-Yao Chang, Taipei City (TW); Chun-Ying Juan, New Taipei City (TW); Kuan-Jung Chen, New Taipei City (TW); Ho-Chung Fu, Kaohsiung City (TW)

(21) Appl. No.: 14/565,403

(22) Filed: Dec. 9, 2014

(51) Int. Cl. A61M 1/00

(52) U.S. Cl. CPC A61M 1/0066

(57) ABSTRACT

A hidden and wearable breast-milk pumping system includes a pumping unit, a collecting unit and a wireless control unit. The pumping unit includes a pumping module and an attaching layer configured for attaching to a breast. The pumping module includes a nozzle, a pumping chamber, a pumping tube connected to the pumping chamber and a milk tube. The nozzle covers the breast and connects the pumping chamber connected between the nozzle and the milk tube. The collecting unit is connected to the milk tube to collect and store the breast milk in the milk tube. The wireless-control unit includes a control module, a vacuum pump and a wireless module. The vacuum pump is connected to the pumping tube. The control module controls the vacuum pump according to a control signal received by the wireless module from a remote device.

### BACKGROUND

1. Technical Field

The invention relates to a breast-milk pumping system, and particularly relates to a hidden and wearable breast-milk pumping system.

2. Related Art

Breast milk can provide baby nutrients and enhance immunity, and is the best natural food and medicine for the baby. In past researches, it is found that the breast milk also has effects of resisting Escherichia coli and Streptococcus infection, and since baby's immune system is still immature, the baby is more susceptible to bacteria than adults. Therefore, doctors agree unanimously that the breast milk is the best food for newborns. However, an office worker after the end of parental leave, or a woman just experienced caesarean section is unable to personally feed the baby at the breast because of work or limited mobility. Now, most mothers would use a breast pump to collect breast milk, and feed the baby with the collected breast milk.

However, most of the breast pumps in the market still require both hands to hold and operate during usage, and although wearable breast pump has appeared in the market, it still requires a sheltered situation for usage, which is quite inconvenience for working woman. Moreover, after a mother uses a breast pump to complete pumping breast milk, the pumped breast milk is required to be further poured into a milk collecting bottle, and such move leads to a contact between the fresh breast milk and the air, and since there are too many unknown bacteria in the air, it is extremely unfavorable to preservation of the breast milk, and even becomes a channel that bacteria infect the baby.

### SUMMARY

The invention is directed to a hidden and wearable breast-milk pumping system, in which a pumping unit, a collecting unit and a wireless control unit thereof are suitable for hiding in outer clothing, and it is unnecessary to use the hidden and wearable breast-milk pumping system at a private and sheltered place.

The invention provides a hidden and wearable breast-milk pumping system suitable for wearing on user's breast, and includes a pumping unit, a collecting unit and a wireless control unit. The pumping unit includes an attaching layer and a pumping module. The attaching layer is attached to a breast and includes an opening. The pumping module includes a nozzle, a pumping chamber, a pumping tube and a milk tube. The nozzle covers the breast and penetrates via the attaching layer through the opening to be connected to the pumping chamber. The pumping chamber is connected between the nozzle and the milk tube. The pumping tube is connected to the pumping chamber for providing a negative pressure to the pumping chamber, and pumps breast milk through the negative pressure to make the breast milk to flow into the milk tube. The collecting unit is connected to the milk tube to collect and store the breast milk in the milk tube. The wireless control unit includes a control module, a vacuum pump and a wireless module. The vacuum pump is connected to the pumping tube for providing the negative pressure. The control module is coupled to the vacuum pump and the wireless module. The wireless module receives a control signal from a remote device through a wireless communication protocol. The control module controls the vacuum pump according to the control signal.

In an embodiment of the invention, the pumping module further includes a liquid-gas separation member, which is disposed in the pumping chamber and located next to the pumping tube, so as to block the breast milk from being pumped into the pumping tube.

In an embodiment of the invention, the liquid-gas separation member includes a block portion and a guide portion, the block portion is configured to block the breast milk from being pumped into the pumping tube, and the guide portion is located between the block portion and the pumping tube for guiding the blocked breast milk to the milk tube.

In an embodiment of the invention, the pumping module further includes a check valve, which is disposed in the pumping chamber to prevent the breast milk from flowing back to the pumping chamber from the milk tube.

In an embodiment of the invention, the collecting unit further includes a fixing mechanism and a collecting bag. The collecting bag is disposed on the fixing mechanism, and the fixing mechanism is detachably fixed on an underclothing of the user, and the milk tube is connected to the collecting bag for injecting the pumped breast milk into the collecting bag.

In an embodiment of the invention, the fixing mechanism includes a first fixing clamp configured to clamp the underclothing, so as to fix the fixing mechanism to the underclothing.

In an embodiment of the invention, the fixing mechanism further includes a second fixing clamp configured to clamp the collecting bag, so as to fix the collecting bag to the fixing mechanism.

In an embodiment of the invention, the collecting unit further includes a sensor, which is disposed on the fixing mechanism for detecting whether the breast milk in the collecting bag reaches a full amount.

In an embodiment of the invention, the sensor is coupled to the wireless control unit, and when the sensor detects that the breast milk in the collecting bag reaches the full amount, the control module controls the vacuum pump to stop providing the negative pressure.

In an embodiment of the invention, the wireless control unit further includes a casing and a silencer cushion member, where the casing is used for accommodating the control module, the vacuum pump and the wireless module, and the silencer cushion member is disposed between the vacuum pump and the casing.

In an embodiment of the invention, a thickness of the pumping module is between 2 cm and 4 cm.

In an embodiment of the invention, a number of the pumping units is two, and the two attaching layers and the two nozzles of the pumping units respectively cover two breasts of the user.

In an embodiment of the invention, the two pumping tubes are respectively connected to the vacuum pump, and the control module controls the vacuum pump to provide the negative pressure to at least one of the two pumping tubes.

In an embodiment of the invention, the control module controls a pumping strength and a pumping cycle provided by the vacuum pump.

In an embodiment of the invention, the remote device includes a smart phone.

According to the above descriptions, the hidden and wearable breast-milk pumping system of the invention is attached to the breast of the user through the attaching layer of the pumping unit, and a volume of the pumping module of the pumping unit is very small, such that the pumping unit can be easily hidden in an outer clothing of the user or covered by a breastfeeding underwear of the user. Moreover, the hidden and wearable breast-milk pumping system further includes the collecting unit, which is connected to the milk tube of the pumping unit for collecting and storing the breast milk from the milk tube. In this way, the breast milk pumped by the pumping unit can be directly stored in the collecting unit without additionally pouring the breast milk into another milk collecting bottle, so as to reduce a risk of polluting the breast milk. In addition, the collecting unit can be fixed to the underwear of the user, such that the pumping unit and the collecting unit can be hidden in the outer clothing. Therefore, it is unnecessary to use the hidden and wearable breast-milk pumping system at a private and sheltered place, so as to improve concealment and usage convenience of the hidden and wearable breast-milk pumping system.

Moreover, the hidden and wearable breast-milk pumping system further includes the wireless control unit, and the wireless module thereof can receive the control signal sent by the remote device through a wireless communication protocol. The control module of the wireless control unit controls a pumping action of the pumping unit according to the control signal. Therefore, the user can control a pumping strength, a pumping magnitude and a pumping cycle of the pumping unit of the hidden and wearable breast-milk pumping system through the remote device, so as to improve usage flexibility and convenience of the hidden and wearable breast-milk pumping system.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a hidden and wearable breast-milk pumping system according to an embodiment of the invention.

FIG. 2 is an exploded view of a part of components of a pumping unit of FIG. 1.

FIG. 3 is a cross-sectional view of the pumping unit of FIG. 1.

FIG. 4 is a partial schematic diagram of a collecting unit of FIG. 1.

FIG. 5 is a schematic diagram of connection between a wireless control unit of FIG. 1 and a remote device.

### DETAILED DESCRIPTION OF DISCLOSED EMBODIMENTS

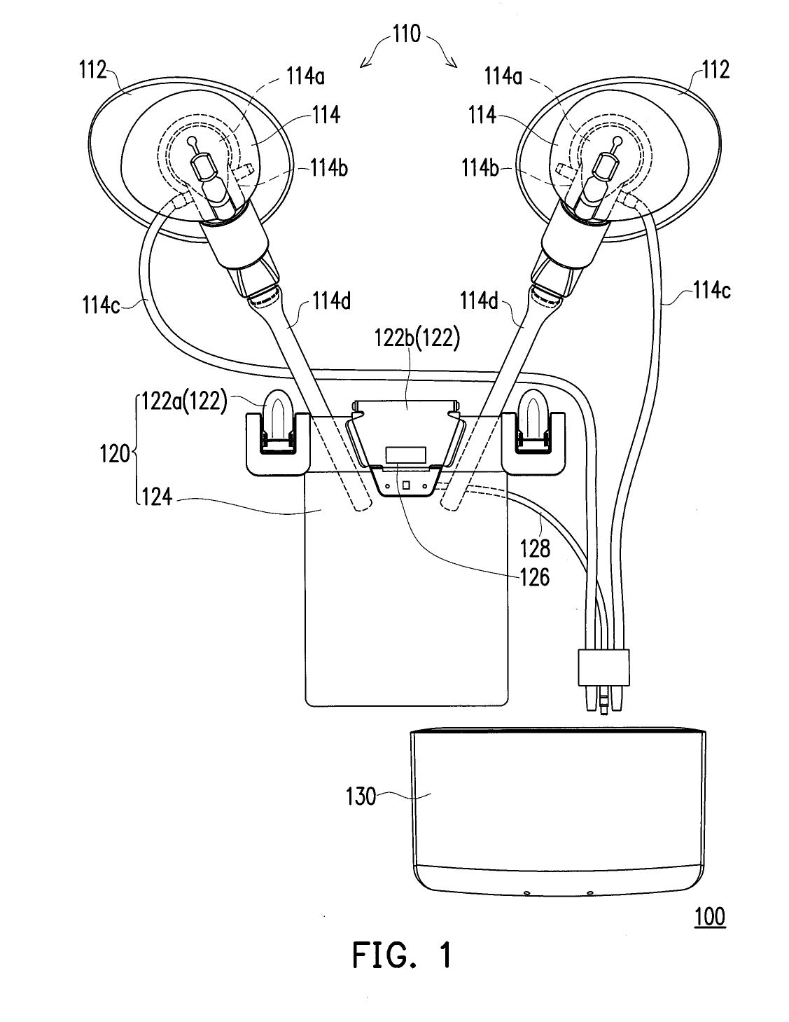

FIG. 1 is a schematic diagram of a hidden and wearable breast-milk pumping system according to an embodiment of the invention. Referring to FIG. 1, the hidden and wearable breast-milk pumping system 100 of the present embodiment is suitable for wearing on user's breasts and hiding in an outer clothing of the user. To be specific, the hidden and wearable breast-milk pumping system 100 can be combined with an underclothing of the user and wear on the user's breasts. In the present embodiment, the underclothing is, for example, breastfeeding underwear. In detail, the hidden and wearable breast-milk pumping system 100 includes at least one pumping unit 110, a collecting unit 120 and a wireless control unit 130. The pumping unit 110 includes an attaching layer 112 and a pumping module 114. In the present embodiment, the number of the pumping units 110 can be two as that shown in FIG. 1, and the attaching layers 112 of the two pumping units 110 respectively covers the two breasts of the user. The attaching layer 112 is attached to the breast of the user and includes an opening 112a. In the present embodiment, a material of the attaching layer 112 is, for example, a skin-friendly attaching material, such as silicone, etc., which can be directly attached to the skin of the user for fixing the pumping module 114 to the user's breast. The attaching layer 112 provides the user a soft and comfortable feeling and is able to withstand a pumping force of the pumping module 114 when the pumping module 114 is pumping the breast milk.

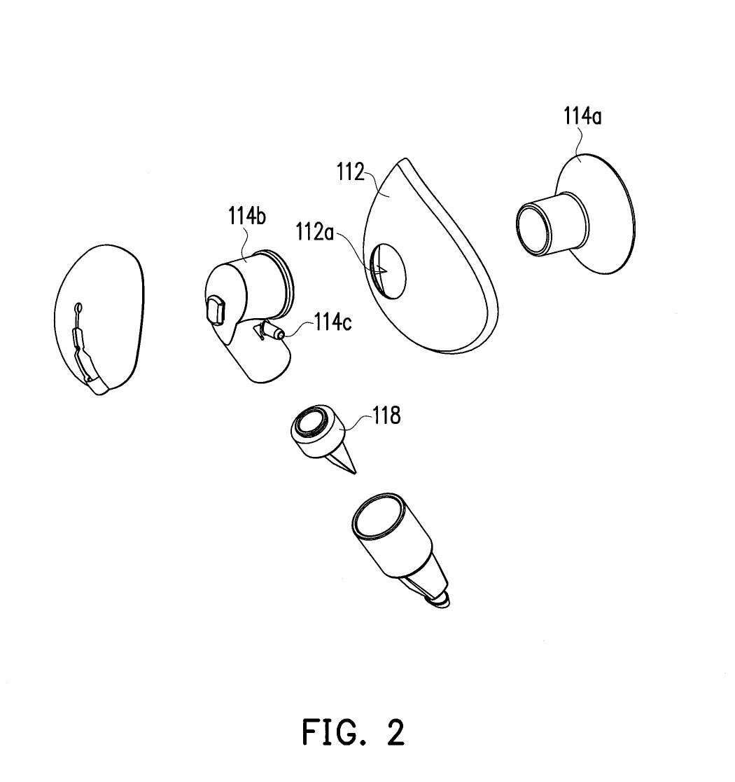

FIG. 2 is an exploded view of a part of components of the pumping unit of FIG. 1. Referring to FIG. 1 and FIG. 2, the pumping module 114 includes a nozzle 114a, a pumping chamber 114b, a pumping tube 114c and a milk tube 114d. The nozzle 114a covers the breast of the user and penetrates through the attaching layer 112 through the opening 112a of the attaching layer 112 to connect the pumping chamber 114b. In detail, the nozzle 114a may include a bell-shaped pumping hood and a connecting tube, where the bell-shaped pumping hood covers the breast of the user, and the connecting tube penetrates through the opening 112a of the attaching layer 112 and is connected to the pumping chamber 114b. Owing to the design of the nozzle 114a and the pumping chamber 114b which are detachably assembled together, not only the pumping module 114 is easy to disassemble for cleaning, but the weight distribution of the pumping module 114 is also more balance since the nozzle 114a and the pumping chamber 114b are located at two opposite sides of the attaching layer 112 after being assembled, so as to avoid the problem of the weight overly concentrated at the side of the attaching layer 112 not attaching to the breast, which results in the attaching layer 112 falling off from the breast by the drag of the weight. Therefore, the nozzle 114a of the embodiment is covered by the attaching layer 112 to be tightly attached to the breast which improves the bonding strength between the pumping unit 110 and the breast. The pumping chamber 114b is connected between the nozzle 114a and the milk tube 114d. The pumping tube 114c is connected to the pumping chamber 114b for providing a negative pressure to the pumping chamber 114b, and pumps breast milk from the breast through the negative pressure to make the breast milk to flow into the milk tube 114d.

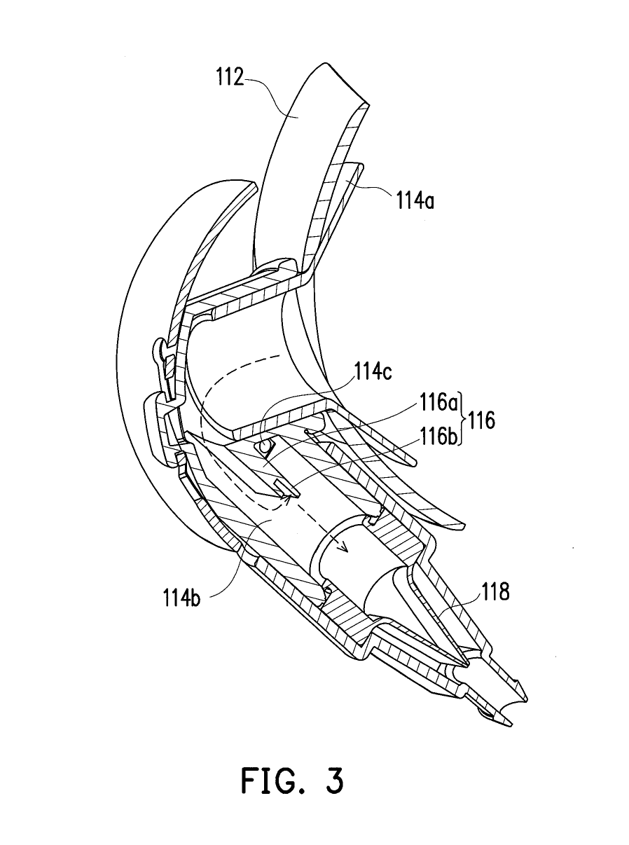

FIG. 3 is a cross-sectional view of the pumping unit of FIG. 1. Referring to FIG. 2 and FIG. 3, in the present embodiment, the pumping module 114 further includes a liquid-gas separation member 116, which is disposed in the pumping chamber 114b and located right next to the pumping tube 114c, so as to block the breast milk from being pumped into the pumping tube 114c. In detail, the liquid-gas separation member 116 can be disposed between the connecting tube of the nozzle 114a and the pumping tube 114c as shown in FIG. 3, and includes a block portion 116a and a guide portion 116b. The block portion 116a is configured to block the breast milk from being pumped into the pumping tube 114c, and the guide portion 116b is located between the block portion 116a and the pumping tube 114c for guiding the breast milk blocked by the block portion 116a to flow into the milk tube 114d shown in FIG. 1.

In the present embodiment, a length of the guide portion 116b can be greater than a length of the block portion 116a, and a flowing direction of the breast milk can be shown as a dotted arrow in FIG. 3, where the breast milk is first drawn by the negative pressure provided by the pumping tube 114c and flows towards the pumping tube 114c, and is then blocked by the block portion 116a. At the time, since the length of the guide portion 116b is greater than the length of the block portion 116a, the breast milk blocked by the block portion 116a can be guided to the milk tube 114d shown in FIG. 1 along an extending direction of the guide portion 116b.

In this way, the liquid-gas separation member 116 may achieve an effect of separating liquid and gas to prevent the breast milk from pumping into the pumping tube 114c while occupying very small space. Moreover, since the volume of the liquid-gas separation member 116 of the present embodiment is very small, a volume of the pumping module 114 is effectively reduced to achieve an effect of miniaturization, such that the pumping unit 110 can be easily hidden in the outer clothing of the user, or covered by the breastfeeding underwear of the user and worn on the user through the breastfeeding underwear. To be specific, a thickness of the pumping module 114 may range between 2 cm to 4 cm. In the present embodiment, the thickness of the pumping module 114 is about 3 cm. Certainly, the present embodiment is only an example, and the invention is not limited thereto. Moreover, the pumping module 114 may further include a check valve 118, which is disposed in the pumping chamber 114b to prevent the breast milk from flowing back to the pumping chamber 114b from the milk tube 114d due to movement of the user. To be specific, the check valve 118 can be disposed at an end portion of the pumping chamber 114b where the milk tube 114d is connected.

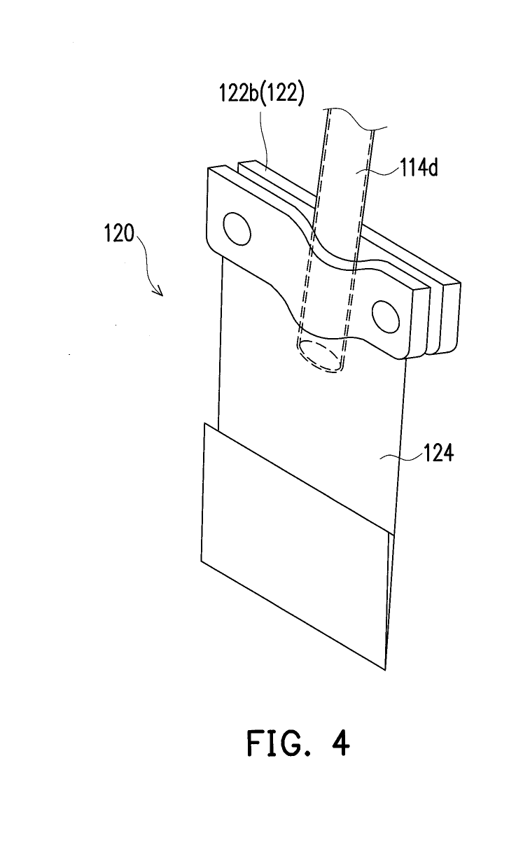

FIG. 4 is a partial schematic diagram of the collecting unit of FIG. 1.

Referring to FIG. 1 and FIG. 4, in the present embodiment, the collecting unit 120 is connected to the milk tube 114d to collect and store the breast milk from the milk tube 114d. To be specific, the collecting unit 120 may include a fixing mechanism 122 and a collecting bag 124. The milk tube 114d can be connected to the collecting bag 124 as that shown in FIG. 4, so as to inject the breast milk pumped by the pumping unit 110 into the collecting bag 124 of the collecting unit 120. The collecting bag 124 is disposed on the fixing mechanism 122, and the fixing mechanism 122 is detachably fixed on the underclothing of the user.

In detail, the fixing mechanism 122 includes at least one first fixing clamp 122a and one second fixing clamp 122b, where the first fixing clamp 122a can be used for clamping the underclothing of the user, so as to fix the fixing mechanism 122 on the underclothing. For example, the pumping unit 110 can be hidden in the breastfeeding underwear of the user, and the first fixing clamp 122 can clamp a lower edge of the breastfeeding underwear of the user to fix the fixing mechanism 122 on the breastfeeding underwear of the user. The second fixing clamp 122b is used for clamping the collecting bag 124 for fixing the collecting bag 124 on the fixing mechanism 122. In the present embodiment, the second fixing clamp 122b can clamp the collecting bag 124 to the fixing mechanism 122 through magnetic adsorption. Certainly, the present embodiment is only an example, and the invention is not limited thereto.

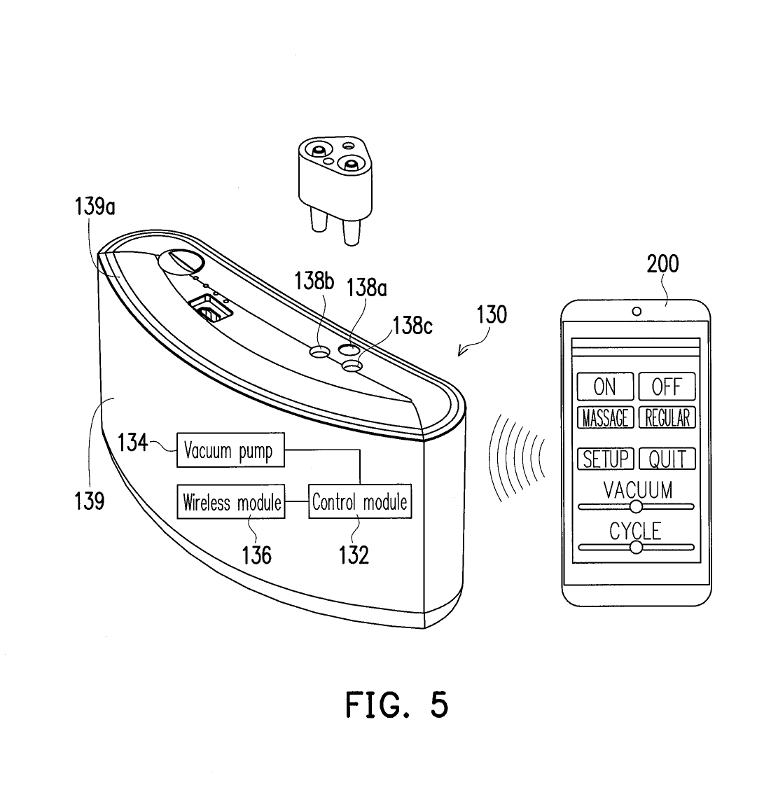

FIG. 5 is a schematic diagram of connection between the wireless control unit of FIG. 1 and a remote device. Referring to FIG. 1 and FIG. 5, in the present embodiment, the wireless control unit 130 includes a control module 132, a vacuum pump 134 and a wireless module 136. The vacuum pump 134 is connected to the pumping tube 114c of the pumping unit 110 for providing the negative pressure. The vacuum pump 134 can be a pumping motor, which provides the negative pressure to the pumping tube 114c and the pumping chamber 114b connected thereto by vacuuming, so as to pump the breast milk by the negative pressure. The control module 132 is coupled to the vacuum pump 134 and the wireless module 136. The wireless module 136 receives a control signal from a remote device 200 through a wireless communication protocol. The control module 132 controls an operation of the vacuum pump 134 according to the control signal, for example, controls a pumping strength, a pumping magnitude and a pumping cycle provided by the vacuum pump 134.

To be specific, the remote device 200 may have a control module, a wireless module and a man-machine interface input device. The wireless module 136 can be connected to a wireless module of the remote device 200 through the wireless communication protocol, so as to receive the control signal sent from the remote device 200. In this way, the user can produce a signal through the man-machine interface input device of the remote device 200, and the control module of the remote device 200 transmits the signal produced by the man-machine interface input device to the control module 132 through the wireless communication protocol, and then the control module 132 controls the operation of the vacuum pump 134 according to the signal, such that after installing specific applications to the remote device 200, the user can control the pumping strength, the pumping magnitude and the pumping cycle through the remote device 200. In the present embodiment, the remote device 200 can be a smart phone or a tablet personal computer (PC), though the invention is not limited thereto.

In the present embodiment, the hidden and wearable breast-milk pumping system 100 may have two pumping units 110 as shown in FIG. 1, and the two pumping tubes 114c of the two pumping units 110 are respectively connected to the vacuum pump 134. The control module 132 controls the vacuum pump 134 to provide the negative pressure to at least one of the two pumping tubes 114c. In other words, the control module 132 can independently control the pumping actions of the two pumping units 110, i.e. the control module 132 can control the vacuum pump 134 to provide the negative pressure to one of the two pumping tubes 114c to perform unilateral breast pump on one breast of the user, or the control module 132 can control the vacuum pump 134 to provide the negative pressure to both of the two pumping tubes 114c to perform bilateral breast pump on both of the two breast of the user.

Moreover, the collecting unit 120 may further include a sensor 126, which is disposed on the fixing mechanism 122 and located corresponding to a bag mouth of the collecting bag 124 for detecting whether the breast milk stored in the collecting bag 124 reaches a full amount. The sensor 126 is coupled to the control module 132. To be specific, the sensor 126 can be a photo coupler. The sensor 126 is coupled to the control module 132 of the wireless control unit 130. In this way, when the sensor 126 detects that the breast milk stored in the collecting bag 124 reaches the full amount, the control module 132 controls the vacuum pump 134 to stop providing the negative pressure, so as to stop the pumping action to avoid overflow of the breast milk.

In the present embodiment, the wireless control unit 130 may further include a casing 139 and a silencer cushion member 139a, where the casing 139 is used for accommodating the control module 132, the vacuum pump 134 and the wireless module 136, and the silencer cushion member 139a can be disposed between the vacuum pump 134 and the casing 139. In the present embodiment, the silencer cushion member 139a surrounds an inner edge of the casing 139 to serve as a cushion between the vacuum pump 134 and the casing 139, so as to reduce noises generated by the vacuum pump 134. Moreover, the casing 139 may further have a sensing line hole 138a and pumping tube holes 138b and 138c (the number of the pumping tube holes corresponds to the number of the pumping tubes 114c), such that a sensing line 128 of the sensor 126 can be coupled to the control module 132 through the sensing line hole 138a, and the pumping tubes 114c can be coupled to the vacuum pump 134 through the pumping tube holes 138b and 138c.

In summary, the hidden and wearable breast-milk pumping system of the invention is attached to the breast of the user through the attaching layer of the pumping unit, and a volume of the pumping module of the pumping unit is very small, such that the pumping unit can be easily hidden in an outer clothing of the user or covered by a breastfeeding underwear of the user, so as to improve concealment and usage convenience of the hidden and wearable breast-milk pumping system. Moreover, the hidden and wearable breast-milk pumping system further includes the collecting unit, which is connected to the milk tube of the pumping unit for collecting and storing the breast milk from the milk tube. In this way, the breast milk pumped by the pumping unit can be directly stored in the collecting unit without additionally pouring the breast milk into another milk collecting bottle, so as to reduce a chance that the breast milk contacts the air and reduce a risk of polluting the breast milk. In addition, the collecting unit can be fixed to the underwear of the user, such that the pumping unit and the collecting unit can be hidden in the outer clothing. Therefore, it is unnecessary to use the hidden and wearable breast-milk pumping system at a private and sheltered place.

Moreover, the hidden and wearable breast-milk pumping system further includes the wireless control unit, and the wireless module thereof can receive the control signal sent from the remote device through a wireless communication protocol. The control module of the wireless control unit controls a pumping action of the pumping unit according to the control signal. Therefore, the user can control a pumping strength, a pumping magnitude and a pumping cycle of the pumping unit of the hidden and wearable breast-milk pumping system through the remote device, so as to improve usage flexibility and convenience of the hidden and wearable breast-milk pumping system.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. A hidden and wearable breast-milk pumping system, comprising:
a least one pumping unit, comprising:
an attaching layer, attached to a breast of a user and comprising an opening; and
a pumping module, comprising a nozzle, a pumping chamber, a pumping tube and a milk tube, wherein the nozzle covers the breast and penetrates through the attaching layer via the opening to be connected to the pumping chamber, the pumping chamber is connected between the nozzle and the milk tube, and the pumping tube is connected to the pumping chamber for providing a negative pressure to the pumping chamber so as to pump breast milk through the negative pressure and make the breast milk flow into the milk tube;
a collecting unit, connected to the milk tube to collect and store the breast milk from the milk tube; and
a wireless control unit, comprising a control module, a vacuum pump and a wireless module, wherein the vacuum pump is connected to the pumping tube for providing the negative pressure, the control module is coupled to the vacuum pump and the wireless module, the wireless module receives a control signal from a remote device through a wireless communication protocol, and the control module controls the vacuum pump according to the control signal.

2. The hidden and wearable breast-milk pumping system as claimed in claim 1, wherein the pumping module further comprises a liquid-gas separation member disposed in the pumping chamber and located right next to the pumping tube, so as to block the breast milk from being pumped into the pumping tube.

3. The hidden and wearable breast-milk pumping system as claimed in claim 2, wherein the liquid-gas separation member comprises a block portion and a guide portion, the block portion is configured to block the breast milk from being pumped into the pumping tube, and the guide portion is located between the block portion and the pumping tube for guiding the blocked breast milk to flow into the milk tube.

4. The hidden and wearable breast-milk pumping system as claimed in claim 1, wherein the pumping module further comprises a check valve disposed in the pumping chamber to prevent the breast milk from flowing back to the pumping chamber from the milk tube.

5. The hidden and wearable breast-milk pumping system as claimed in claim 1, wherein the collecting unit further comprises a fixing mechanism and a collecting bag, the collecting bag is disposed on the fixing mechanism, and the fixing mechanism is detachably fixed to an underclothing of the user, and the collecting bag is connected to the milk tube for collecting and storing the breast milk from the milk tube.

6. The hidden and wearable breast-milk pumping system as claimed in claim 5, wherein the fixing mechanism comprises a first fixing clamp configured to clamp the underclothing, so as to fix the fixing mechanism to the underclothing.

7. The hidden and wearable breast-milk pumping system as claimed in claim 6, wherein the fixing mechanism further comprises a second fixing clamp configured to clamp the collecting bag, so as to fix the collecting bag to the fixing mechanism.

8. The hidden and wearable breast-milk pumping system as claimed in claim 1, wherein the collecting unit further comprises a sensor disposed on the fixing mechanism for detecting whether the breast milk in the collecting bag reaches a full amount.

9. The hidden and wearable breast-milk pumping system as claimed in claim 8, wherein the sensor is coupled to the wireless control unit, and when the sensor detects that the breast milk in the collecting bag reaches the full amount, the control module controls the vacuum pump to stop providing the negative pressure.

10. The hidden and wearable breast-milk pumping system as claimed in claim 1, wherein the wireless control unit further comprises a casing and a silencer cushion member, the casing is used for accommodating the control module, the vacuum pump and the wireless module, and the silencer cushion member is disposed between the vacuum pump and the casing.

11. The hidden and wearable breast-milk pumping system as claimed in claim 1, wherein a thickness of the pumping module is substantially between 2 cm and 4 cm.

12. The hidden and wearable breast-milk pumping system as claimed in claim 1, wherein a number of the at least one pumping unit is two, and the two attaching layers and the two nozzles of the pumping units respectively cover two breasts of the user.

13. The hidden and wearable breast-milk pumping system as claimed in claim 12, wherein the two pumping tubes are respectively connected to the vacuum pump, and the control module controls the vacuum pump to provide the negative pressure to at least one of the two pumping tubes.

14. The hidden and wearable breast-milk pumping system as claimed in claim 1, wherein the control module controls a pumping strength and a pumping cycle provided by the vacuum pump.

15. The hidden and wearable breast-milk pumping system as claimed in claim 1, wherein the remote device comprises a smart phone.
